(19) United States
(12) Patent Application Publication
(10) Pub. No.: US 2015/0023935 A1
(43) Pub. Date: Jan. 22, 2015

(54) Reprogramming of Aged Adult Stem Cells

(71) Applicant: Advanced ReGen Medical Technologies, Inc., Montclair, NJ (US)

(72) Inventor: Vincent C. Giampapa, Montclair, NJ (US)

(21) Appl. No.: 14/509,523

(22) Filed: Oct. 8, 2014

Related U.S. Application Data
(63) Continuation-in-part of application No. 13/785,691, filed on Mar. 5, 2013.
(60) Provisional application No. 61/608,480, filed on Mar. 8, 2012.

(51) Int. Cl. C12N 5/0789; C12N 15/01

(52) U.S. Cl. CPC C12N 5/0647; C12N 15/01; C12N 2510/00
USPC 424/93.21

(57) ABSTRACT

A method of reprogramming aged human or animal adult stem cells (AASC), comprising the steps of (a) collecting young adult stem cells (YASC) from the blood of a donor wherein the donor is less than 40 years old; (b) dissolving at least a portion of membranes of said YASC to release an intracellular matrix (ICM) thereof; (c) applying a supernatant of said ICM to a culture of AASC; and (d) following exposure of said AASC to said supernatant for a bioactively sufficient period, infusing such an exposed AASC to the donor thereof. A method comprising the steps of (a) collecting young adult stem cells (YASC) from the blood of a donor wherein the donor is less than 40 years old; (b) dissolving at least a portion of membranes of said YASC to release an intracellular matrix (ICM) thereof; (c) applying a supernatant of said ICM to a culture of aged adult stem cells (AASC) to produce exposed AASC; (d) recovering at least a portion of the exposed AASC; and (e) introducing the exposed AASC to the donor wherein the donor is greater than 40 years of age and is diagnosed with a disorder selected from the group consisting of AIDS, an age-related disease, and an immune deficiency disorder.

### CROSS-REFERENCE TO RELATED APPLICATIONS

The present application is a continuation-in-part of and claims priority to U.S. patent application Ser. No. 13/785,691, filed on Mar. 5, 2013, published as U.S. 2013/0236428 and entitled “Reprogramming of Aged Adult Stem Cells,” which is incorporated by reference herein in its entirety and claimed the benefit under 35 USC 119 (e) of the U.S. Provisional Patent Application Ser. No. 61/608,480, filed Mar. 8, 2012, which is also hereby incorporated by reference in its entirety.

### TECHNICAL FIELD

The present disclosure relates to methods of reprogramming aged adult stem cells.

### BACKGROUND

Adult stem cells normally age with each decade and decrease in number and functional quality. This contributes to aging and loss of function of the human body as well as contributes to the diseases of aging like diabetes, arthritis, cardiovascular disease, and decreased immune function.

The instant invention is an improvement in the field because no methods exist to improve the function of previously collected and stored old adult stem cells using unaltered young stem cell fluid and cellular components, with or without, oocyte of fetal component retreatment.

This invention focuses on reprogramming the genes involved in youthful stem cell function within cells that have been previously collected and stored using a number of techniques including apheresis, bone marrow aspiration, or other accepted collection methods. The cells of persons who are 40 years and older have multiple functional-cellular defects, due to the cellular aging process, that can be corrected by using the natural, unaltered young cell fluid and cellular components of young adult stem cells.

The relevant prior art, as known to the inventor, includes a May 2011, publication in FASEB. 5-25. 1474-1485 (2011) by Sun et al., entitled Rescuing Replication and Osteogenesis of Aged Mesenchymal Stem Cells by Exposure to Young Extracellular Matrix; PCT Publication WO 2004/048555 A1 to Millar et al., entitled Restoration of Methylation States in Cells; and PCT Publication WO 2007/016245 A2 to Fitzsimmons et al, entitled Reprogramming of Adult or Neonic Stem Cells and Methods of Use.

### SUMMARY

Disclosed herein is a method of reprogramming aged human or animal adult stem cells (AASC), comprising the steps of (a) collecting young adult stem cells (YASC) from the blood of a donor wherein the donor is less than 40 years old; (b) dissolving at least a portion of membranes of said YASC to release an intracellular matrix (ICM) thereof; (c) applying a supernatant of said ICM to a culture of AASC; and (d) following exposure of said AASC to said supernatant for a bioactively sufficient period, infusing such an exposed AASC to the donor thereof.

Also disclosed herein is a method comprising the steps of (a) collecting young adult stem cells (YASC) from the blood of a donor wherein the donor is less than 40 years old; (b) dissolving at least a portion of the membranes of said YASC to release an intracellular matrix (ICM) thereof; (c) applying a supernatant of said ICM to a culture of aged adult stem cells (AASC) to produce exposed AASC; (d) recovering at least a portion of the exposed AASC; and (e) introducing the exposed AASC to the donor wherein the donor is greater than 40 years of age and is diagnosed with a disorder selected from the group consisting of AIDS, an age-related disease, and an immune deficiency disorder.

### BRIEF DESCRIPTION OF THE DRAWINGS

FIG. 1 is a conceptual flow diagram of the inventive method showing the use of the supernatant of young adult cells to reprogram aged adult stem cells and, optionally, the use of oocytes and fetal factors therewith.

### DETAILED DESCRIPTION

In an embodiment, a method of AASC reprogramming comprises the steps of (a) collecting YASC from the blood of a human donor, using apheresis or like means; (b) using normal diffusion, cell lysis, or like means to at least partially dissolve membranes of said YASC to release an intracellular matrix (ICM) thereof; (c) applying a supernatant of said YASC to a culture of AASC to be reprogrammed; and (d) following exposure of said AASC to said supernatant for a bioactively sufficient period, infusing such an exposed AASC to the donor thereof, either with or without cell expansion.

Disclosed herein are methods of reprogramming of mammalian and/or AASC with the supernatant-intracellular matrix, bioactive lipids, and/or microvesicles of YASC in a single step. In some embodiments, methods of reprogramming of mammalian and/or AASC with the supernatant-intracellular matrix, bioactive lipids, and/or microvesicles of YASC further comprise utilizing oocyte supernatant, its intracellular matrix, and/or cellular components to accomplish gene erasure and reprogramming. This invention focuses on reprogramming and/or reactivating genes that are active and involved in youthful adult stem cell function, within AASC that have been previously collected by and/or stored for patients who are 40 years old or more. The process is accomplished by using the natural, unaltered YASC fluid and by apheresis of its cellular components. It is an object of the invention to rejuvenate AASC by exposure to the intracellular matrix of YASC, oocytes and/or fetal factors.

As stated above, adult stem cells normally age with each decade and decrease in number and functional quality. This contributes to the human aging process and loss of function of the human organs and body. Such aging occurs at an epigenetic level in which important promoter regions of DNA progressively turn off or cease to function optimally. The invention claimed here solves this problem by enhancing the function of older adult stem cells by exposing the AASC to the cellular products and cell components of YASC, oocytes and/or fetal factors, each of which have different cellular products and cell membrane components.

Reprogramming of AASC with the supernatant of YASC or its ICM or with oocyte supernatant or its ICM, reprograms and reactivates promoter regions of aged genes to a more youthful profile that are involved in maintaining youthful stem cell function. This occurs by “turning off” certain age-related genes and at the same time “turning back on” other genes. This results in a more efficient body cell replacement, turnover, and improved quality of health at the cellular, higher tissue, and body levels.

The claimed invention differs from what currently exists. The present techniques involve use of viral vectors and gene splicing technologies, which are fraught with potential problems including, but not limited to, abnormal cell growth, methodologies involving multiple steps, and questions with regard to the reproducibility of the results. Additionally, these processes are typically expensive and labor intensive.

There presently does not exist any effective clinical method of improving the function of previously collected and stored human AASC using the ICM of young stem cells or the ICM of oocytes, so that such cells can be used to reduce human aging and improve the quality of health as humans grow older. There presently does not exist any clinical method of enhancing the function of previously collected and stored AASC, using the ICM of YASC or the ICM of oocytes, so that such cells can be used to improve human aging and quality of health without the use of complicated cellular manipulation. The methodologies disclosed herein may permit stem cells to be used for clinical intervention as a means of maintaining health and normal function in aging humans.

The past systems of stem cell manipulation did not focus on gene reprogramming of specific genes, or groups of genes, involved with youthful stem cell function by using YASC intracellular fluid and components, either with or without oocyte cell fluid, or cell components.

This invention focuses on reprogramming the genes involved in youthful adult stem cell function, within the cells that have been previously collected and stored for patients who are 40 years and older, by using natural, unaltered young cell fluid and its components.

The invention discussed relates to individual cell components which include but are not limited to:
amino acids
bioactive lipids
chemokines
culture media
DNA
hormonal compounds
iRNA
microvesicles
micro-RNA
mRNA
old stem cells
oocytes
oocyte cell fluid components
peptides
polypeptides
RNA
transcription factors
various growth factors, and
young stem cells.

### Relationship Between Cellular Components

The invention's active reprogramming components are contained within the YASC cellular components as a natural mixture of the ICM. The microvesicles are part of the cellular membrane itself.

The first step in this method is to place previously collected AASC, after using an apheresis collection process with NEUPOGEN® filgrastim as a mobilizing agent, on a standard culture media or a cell culture dish, which exposes the AASC to the intracellular and membrane components of the YASC. The YASC components may be released by any number of standard cell rupture (lysis) techniques or by simple diffusion. In this fashion, the soluble factors, cell membrane components, and microvesicles are able to diffuse or transfer themselves passively through the AASC membrane and initiate the gene reprogramming effects on appropriate promoter regions of DNA of the AASC.

Without wishing to be limited by theory, reprogramming of the AASC genes occurs through direct exposure of the AASC to the intracellular and membrane components of YASC. The cells to be treated comprise AASC placed on a culture dish and separated from the YASC by a permeable membrane that allows the YASC' soluble factors and bioactive lipids of the cell membrane to cross over to and be transferred directly into and through the cell membrane of the AASC. Once this has occurred, the promoter regions of the genes of the AASC are reprogrammed by the YASC' cellular components after an adequate time of exposure to the YASC' cellular elements. It is contemplated that most of the gene reprogramming occurs at the level of the chromosome, the epigenetic level, and through the processes of methylation, acetylation, and/or phosphorylation. This process can be completed as described in Step One, or a more complete reprogramming can be accomplished in two or three steps.

If a more complete “gene reprogramming” is desired, then the AASC are first exposed to oocytes' cellular fluid components, via the same process as described above, to complete what can be termed “gene imprinting erasure.” The “erasure process” is accomplished because oocytes contain different intracellular soluble factors that do not strictly reprogram but “erase” certain methylation patterns and other epigenetic markers of the AASC. In this more complete process, the “genetically erased” AASC are subsequently treated by exposure to the YASC supernatant, its soluble factors, and cell membrane components (microvesicles), as described previously herein, to reprogram its genes to a similar profile found in the YASC.

### Clinical Procedure: Step One

In the complete process, the procedure can begin by placing the AASC in the presence of an oocyte and allowing the intracellular components from this group of cells to passively transfer through a dividing membrane and into the AASC. This removes some or all of the “genetic programming” or gene imprinting in the AASC. After this process is completed, the original, old gene imprinting is removed or erased and the AASC genes are now ready to be reprogrammed.

### Clinical Procedure: Step Two

This step involves placing the AASC with the “erased genes” into the presence of the YASC solution consisting of, but not limited to, the soluble factors and microvesicle biolipids, and allowing appropriate time for the old, cleaned genes to be reprogrammed by normally occurring diffusion of these elements through the AASC membrane. The diffusion process can be aided by a number of standard cell diffusion enhancement techniques. During this step in the process, the cells are not expanded or multiplied. The elements in this invention that are optional are the oocyte, gene erasure step, or fetal fluid exposure process, and the exposure to their intracellular components, i.e., the gene cleaning or “gene imprinting erasure” step may be optional.

The YASC factors and bioactive lipids interact with the genes of the AASC to affect the reprogramming process at the epigenetic level. For the invention to work more efficiently, the factors and bioactive lipids could be taken from a genetically-related human subject like a son or daughter, but this is not a necessary factor to render the process effective. Markers that are used to identify and confirm the identity of YASC are beta-galactosidase, telomerase, and colony forming units. These markers and factors may be measured prior to exposure to the AASC to be reprogrammed and can be premeasured and/or remeasured after the reprogramming process to document its effectiveness. The process described can be further enhanced by the use of fetal factors and therein biosoluble lipids which can be obtained from umbilical cord stem cells or the amniotic fluid related to birth. Such cells include mesenchymal stem cells, adipose-derived stem cells, stromal cells, skeletal muscle stem cells, neural stem cells, cardiac stem cells, and amniotic fluid cells.

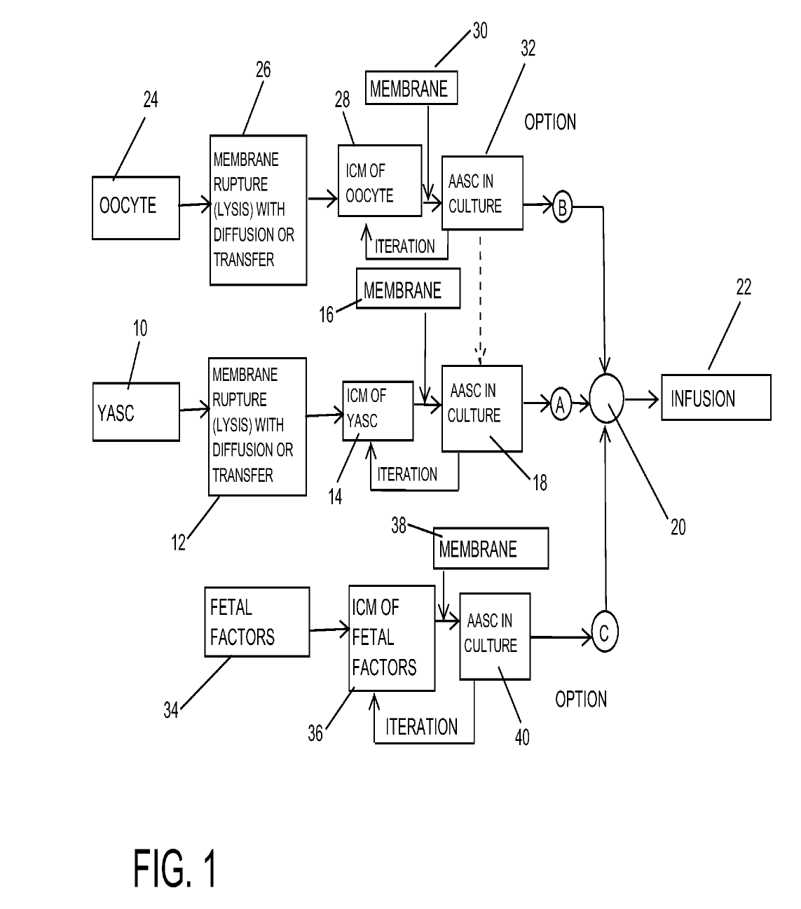

With reference to FIG. 1, there is shown a flow diagram which is an embodiment of the present method of AASC reprogramming, illustrating therein the primary method involving the use of YASC as well as two optional or enhancement methods thereof which, respectively, entail the use of oocytes and fetal factors or amniotic fluid cells. More particularly, looking at the central row in FIG. 1, the process begins with the collection of YASC 10 by any of a number of accepted stem cell collection techniques including, but not limited to, apheresis processes. The collected YASC are then, at step two, subjected to membrane rupture, known as lysis, using any of a number of known processes such as the use of biological detergents or simple diffusion. Such lysis enables diffusion or transfer of the intracellular elements of the stem cells, known as the ICM, as step 14. Therefrom, the ICM passes through a membrane 16, the purpose of which is to allow selective filtration of the desired intracellular components or membrane components of the YASC. The ICM components passing through membrane 16 then are exposed to a culture of the AASC to be reprogrammed, shown at step 18. If this is the sole strategy employed in a given AASC reprogramming project, the culture of step 18 is collected at Step A, concentrated at step 20 and infused, at step 22, into the donor of the AASC. Cell expansion can then be accomplished at this stage prior to using the cells for therapy.

In the event that an oocyte 24 is employed, prior to above-described steps 10 to 18, for purposes of “cleaning” of age-related characteristics of the AASC, the oocyte is then subject to lysis using a method suitable to the membrane thereof, this shown at step 26. The ICM of the oocyte is then employed at step 28 and passes through membrane 30 to form a culture 32 in combination with the AASC. Both steps 18 above and said step 32 may be reiterated relative to steps 14 and 28 respectively several times in order to assure maximum potency of the resultant concentrate B.

As a further option, intended to enhance the effectiveness of the present method, fetal factors 34 may be employed from which the ICM 36 are extracted. These then pass through membrane 38 and are added to the AASC in culture at step 40. The concentrate thereof (indicated by letter C) is mixed at the aggregate concentrate step 20 and then infused into the donor.

As may be seen in FIG. 1, AASC are exposed to the supernatant of YASC (step 18) on a standard cell culture media, in vitro, with or without use of a semipermeable membrane 16 between them. The AASC are then left in contact with the supernatant of the YASC (containing the soluble compounds and microvesicles) for a specific amount of time and/or through serial exposures, to avoid dilution by the AASC components. This process allows the AASC genes to be “reprogrammed,” that is, promoter regions of some genes are “turned off” while others are “turned on.” This process results in a more youthful gene profile resulting in a functionally “reprogrammed old adult stem cell.”

The inventive method can also produce a new class of cell products for intravenous infusion that can be utilized for treating the aging process in general, treating immune deficiencies, treating AIDS patients, and/or treating a general class of age related disease.

The invention can be used topically for wound healing with these reprogrammed cells, treating aging skin to prevent and remove wrinkles, and the treatment of skin cancers.

The reprogrammed cells can be used in experimental lab studies for in vitro cell studies with other stem cells as a comparison to see the effects of other gene expression modifiers.

While there has been shown and described above the preferred embodiment of the instant invention, it is to be appreciated that the invention may be embodied otherwise than is herein specifically shown and described and that, within said embodiment, certain changes may be made in the form and arrangement of the parts without departing from the underlying ideas or principles of this invention as set forth in the claims appended herewith.

What is claimed:

1. A method of reprogramming aged human or animal adult stem cells (AASC), comprising the steps of:
(a) collecting young adult stem cells (YASC) from the blood of a donor wherein the donor is less than 40 years old;
(b) dissolving at least a portion of membranes of said YASC to release an intracellular matrix (ICM) thereof;
(c) applying a supernatant of said ICM to a culture of AASC; and
(d) following exposure of said AASC to said supernatant for a bioactively sufficient period, infusing such an exposed AASC to the donor thereof.

2. The method as recited in claim 1, further comprising reiterating said steps (b) and (c).

3. The method as recited in claim 1, further comprising:
separating said YASC from said supernatant of said YASC by use of a membrane and permitting YASC soluble factors and related components to pass therethrough prior to application of said step (c).

4. The method as recited in claim 3, further comprising:
reiterating said steps (b) and (c).

5. The method as recited in claim 1, further comprising the steps of:
(e) prior to said step (a), collecting oocytes biologically compatible with said AASC to be reprogrammed;
(f) using lysis or like means to dissolve or remove membranes of said oocytes to remove the ICM thereof; and
(g) applying said ICM of said oocytes to said culture of said AASC.

6. The method as recited in claim 5, further comprising the steps of reiterating said steps (b) and (c).

7. The method as recited in claim 1, further comprising:
applying fetal cell products or by-products together with or after said step (c).

8. The method as recited in claim 5, further comprising the step of:
applying fetal cell products or by-products together with or after said step (c).

9. The method of claim 7 wherein the fetal cell products or by-products are obtained from umbilical cord stem cells, amniotic fluid, or both.

10. The method of claim 1 wherein the donor of the YASC and the donor of the AASC are related.

11. The method of claim 1 wherein the YASC are analyzed for beta-galactosidase activity, telomerase activity, or both.

12. The method of claim 1 wherein at least a portion of the exposed AASC is introduced to the donor thereof by intravenous infusion.

13. The method of claim 12 wherein the donor is being treated for an immune deficiency disorder.

14. The method of claim 12 wherein the donor is being treated for AIDS.

15. The method of claim 12 wherein the donor is being treated for an age-related disease.

16. The method of claim 1 wherein at least a portion of the exposed AASC is introduced to the donor thereof by topical application.

17. The method of claim 16 wherein the topical application is applied to a donor for wound healing.

18. The method of claim 16 wherein the topical application is applied to a donor for treatment of aging skin.

19. The method of claim 16 wherein the topical application is applied to a donor for treatment of cancer.

20. A method comprising the steps of:
(a) collecting young adult stem cells (YASC) from the blood of a donor wherein the donor is less than 40 years old;
(b) dissolving at least a portion of membranes of said YASC to release an intracellular matrix (ICM) thereof;
(c) applying a supernatant of said ICM to a culture of aged adult stem cells (AASC) to produce exposed AASC;
(d) recovering at least a portion of the exposed AASC; and
(e) introducing the exposed AASC to the donor wherein the donor is greater than 40 years of age and is diagnosed with a disorder selected from the group consisting of AIDS, an age-related disease, and an immune deficiency disorder.
